(19) United States
(12) Patent Application Publication
(10) Pub. No.: US 2020/0171124 A1
(43) Pub. Date: Jun. 4, 2020

(54) TOPICAL COMPOSITIONS FOR OPHTHALMIC AND OTIC USE

(71) Applicant: Lupin Limited, Mumbai (IN)

(72) Inventors: Sajeev Chandran, Pune (IN); Ashok Bhagwat Nighute, Pune (IN); Makarand Krishnakumar Avachat, Pune (IN)

(21) Appl. No.: 16/093,224

(22) PCT Filed: Apr. 13, 2017
(86) PCT No.: PCT/IB2017/052135

§ 371 Date: Oct. 12, 2018

(30) Foreign Application Priority Data
Apr. 15, 2016 (IN) 201621013287

(51) Int. Cl. A61K 38/14; A61K 9/00; A61K 31/165; A61K 31/407; A61K 9/127; A61K 31/192; A61K 31/196

(52) U.S. Cl. CPC A61K 38/14; A61K 9/0046; A61K 31/165; A61K 31/196; A61K 9/127; A61K 31/192; A61K 9/0048; A61K 31/407

(57) ABSTRACT

The invention relates to topical compositions of lipoglycopeptides. The compositions may additionally contain one or more non-steroidal anti-inflammatory agents. The compositions are used to treat ophthalmic or otic conditions.

### FIELD OF THE INVENTION

The present invention relates to topical compositions of lipoglycopeptides and pharmaceutically acceptable excipients. The lipoglycopeptides of the present invention may be selected from Telavancin, Dalbavancin and Oritavancin. The compositions of the present invention may additionally contain anti-inflammatory agents. The compositions can be used to treat patients suffering from eye and ear infections, particularly those resistant to conventionally used ophthalmic and otic antibiotics.

### BACKGROUND

Telavancin hydrochloride or N3″-[2-(decylamino)ethyl]-29-[[(phosphono-methyl)-amino]-methyl]-hydrochloride is a lipoglycopeptide antibacterial that is a synthetic derivative of vancomycin and is represented by formula:

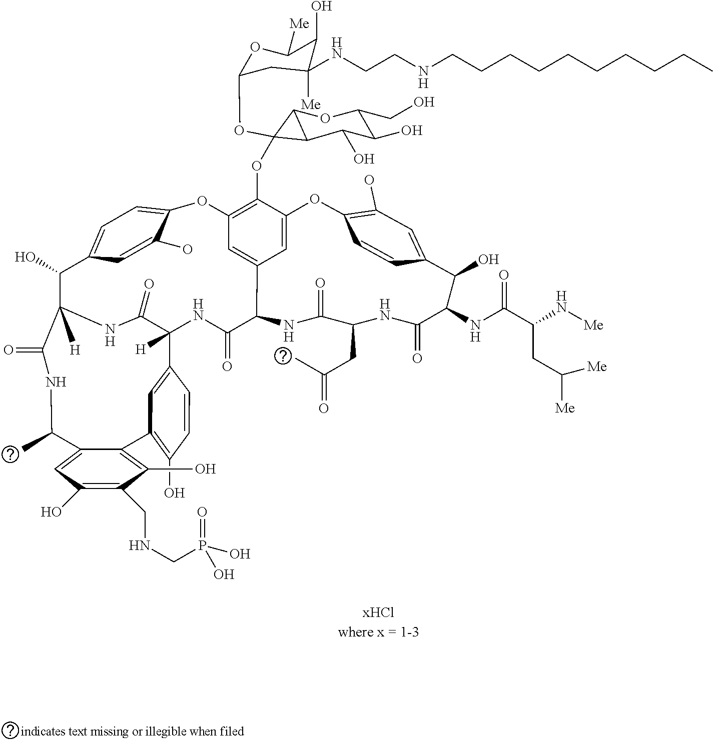

Telavancin (Vibactiv÷) was first approved in US for complicated skin and skin structure infections (cSSSI) and Hospital-acquired and Ventilator-associated bacterial pneumonia in 2009.

U.S. Pat. No. 6,635,618 discloses Telavancin as novel phosphonate derivative of glycopeptide for effective anti-bacterial use. The compositions of Telavancin are described for oral, parenteral and transdermal use.

U.S. Pat. No. 6,858,584 & U.S. Pat. No. 8,158,580 disclose pharmaceutical compositions and specifically detail the parenteral compositions.

U.S. Pat. Nos. 6,872,701, 7,268,971, 7,008,923, 7,208,471, 7,351,691, 7,700,550 & U.S. Pat. No. 8,101,575 disclose oral, parenteral, rectal and transdermal preparation of Telavancin for treating bacterial infections.

Dalbavancin or 5,31-dichloro-38-de(methoxycarbonyl)-7-demethyl-19-deoxy-56-O[2-deoxy-2-[(10-methyl undecanoyl)amino]- [symbol] -D-glucopyranuronosyl]-38-[[3-(dimethylamino)propyl]carbamoyl]-42-O- -D-mannopyranosyl-15-N-methyl(ristomycin A aglycone) hydrochloride is lipoglycopeptide synthesized from a fermentation product of Nonomuraea species and is a mixture of five closely related active homologs (A₀, A₁, B₀, B₁, and B₂). Dalbavancin is represented by formula:

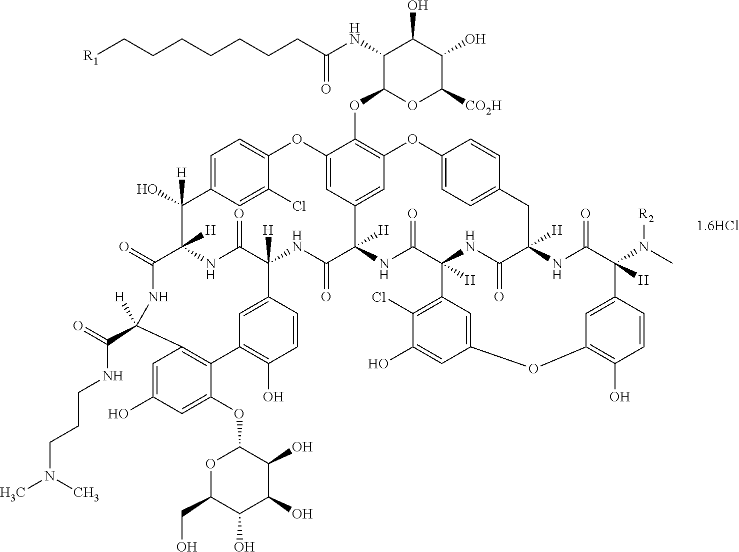

| Dalbavancin | R₁ | R [symbol] |
|---|---|---|
| A [symbol] | CH(CH₃)₂ | H |
| A₁ | CH₂CH₂CH₃ | H |
| B [symbol] | CH [symbol] CH(CH₃)₂ | H |
| B₁ | CH₂CH₂CH₂CH₃ | H |
| B₂ | CH₂CH(CH₃)₂ | CH₃ |

[symbol]

indicates data missing or illegible when filed

Dalbavancin (DALVANCE÷) is approved for acute bacterial skin and skin structure infections (ABSSSI) in year 2014 as powder for intravenous administration (500 mg base).

U.S. Pat. No. 5,750,509 discloses Dalbavancin as amine derivative of glycopeptide for the treatment of bacterial infections.

U.S. Pat. No. 6,900,175 describes a method of treating skin and soft tissue infection using Dalbavancin. The invention discloses that the compositions are administered by parenteral, e.g., intravenous or intramuscular, routes.

U.S. Pat. No. 7,115,564 & U.S. Pat. No. 7,119,061 describe a reconstitutable parenteral dosage form for Dalbavancin, which comprises Dalbavancin along with mannitol and lactose which act as stabilizers for the formulation.

U.S. Pat. No. 8,143,212 discloses method for treatment of bacterial infection by administering initial and subsequent doses of Dalbavancin. The compositions of this invention are lyophilized powders for parenteral use which are to be reconstituted at the time of administration, Oritavancin or [4_R]-22-O-(3-amino-2,3,6-trideoxy-3-C-methyl- -Larabino-hexopyranosyl)-N3 ̆ ̆-[(4 ̆-chloro[1,1 ̆-biphenyl]-4-yl)methyl] vancomycin phosphate is a semisynthetic lipoglycopeptide antibacterial drug represented by formula:

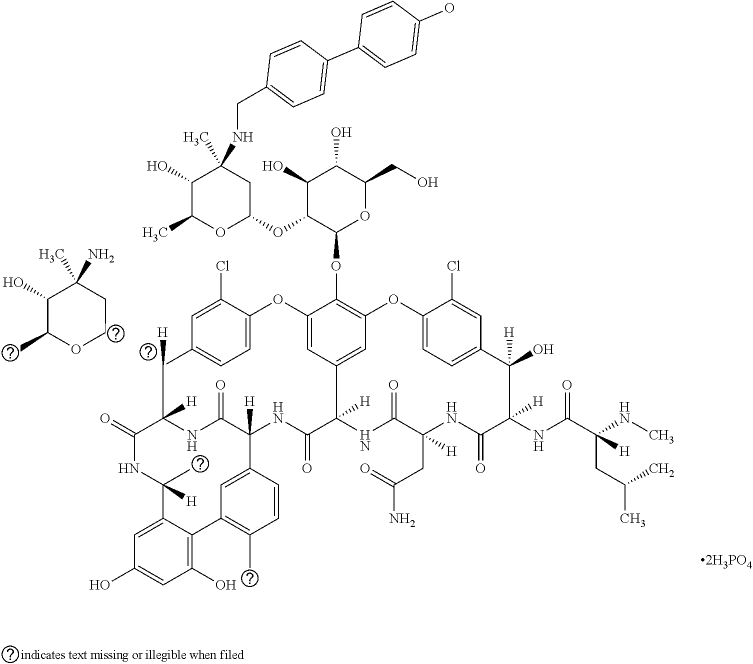

Oritavancin (ORBACTIV÷) was first approved for marketing in US for acute bacterial skin and skin structure infections (ABSSSI) in 2014. It is available as powder for intravenous administration (400 mg base/vial).

U.S. Pat. No. 5,840,684 describe semisynthetic glycopeptide derivative compounds, including Oritavancin having activity against wide variety of gram positive bacteria including vancomycin resistant strains. Pharmaceutical compositions disclosed are oral and parenteral. The patent discloses that Oritavancin can be admixed with conventional pharmaceutical carriers and excipients and used in the form of tablets, capsules, elixirs, suspensions, syrups, wafers, and the like. The patent discloses intramuscular and intravenous compositions as examples for parenteral. Working examples disclose specific oral formulations like capsule, suspension and tablet.

U.S. Pat. No. 8,420,592 discloses the method of use of Oritavancin for the treatment of complicated skin and skin structure infection (cSSSI). The patent discloses that the compositions are administered by parenteral or oral route.

Eye infections are result of invasion of bacteria, fungi and vi ruses. Most commonly infected eye tissues include cornea and conjunctiva. Inflammation is a usual manifestation of all ophthalmic and otic infections. Although the ophthalmic and otic infections are associated with a srmall number of bacteria; the rates of resistance to available antibiotics are rising and differ from strain to strain. Ophthalmic infections due to Methicillin Resistant Staphylococcus (MRS) strains are becoming increasingly prevalent. In United States MRS strains are responsible for over 30% of serious ophthalmic infections. It is worth noting that over 80% of these MRS strains are resistant to all fluoroquinolones, a class of antibiotics used by ophthalmologists worldwide. A part from fluoroquinolones, these strains exhibit resistant to synthetic penicillins (methicillin, oxacillin, naficillin, cloxacillin, dicloxacillin and amoxicillin) and cephalosporins. Conjunctivitis is the most commonly reported manifestation of Methicillin Resistant Staphylococcus aureus (MRSA). Conjunctivitis is an infection of outermost layer of the sclera of the eye and the inner surface of the eyelid. The other conditions include MRSA infectious scleritis, MRSA keratitis, MRSA dacryocystitis, preseptal cellulitis, corneal ulcers, endophthalmitis; orbital cellulitis; blebitis; blepharoconjuctivitis, postoperative MRSA infection. Usually reported ear infections include otitis externa and otitis media. Ruptured tympanic membrane or surgical procedures like tympanostomy risk the middle ear to infections.

Inflammation is a common manifestation of ophthalmic and otic infections. Glucocorticoids are generally used in combination with antibiotics to treat inflammation associated with eye or ear infections. The glucocorticoids, however, are associated with serious side effects e.g., diabetes, suppression of the hypothalamic-pituitary-adrenal axis, osteoporosis etc. Glucocorticoids are frequently used by ophthalmologists for indications which include conjunctivitis, keratitis, scleritis and post-operative management. Adverse effects on eyes specifically include cataract glaucoma, retinal emboli and maculopathy. Corticosteroids may induce ocular hypertension and glaucoma and pose serious risks after local or systemic administration. Improper use of topical corticosteroid in presence of corneal infections may lead to ocular morbidity. Ocular complications of local administration of corticosteroids include ocular surface epithelial toxicity, delayed epithelial healing, reduced wound strength, keratocyte apoptosis, corneal deposits, exacerbation of microbial infection, reactivation of herpes simplex keratitis, crystalline keratopathy, corticosteroid-induced ocular hypertension/glaucoma, corticosteroid-induced posterior subcapsular cataract, lidptosis, dilated pupil extraocular muscle imbalance, orbital fat atrophy, intraocular vascular occlusion/haemorrhage, accidental intraocular penetration, systemic absorption, systemic glucocorticoid suppression.

Semisynthetic lipoglycopeptides like Telavancin, Dalbavancin and Oritavancin promise the treatment of patients with infections caused by multi-drug-resistant Gram-positive pathogens. These compounds inhibit the synthesis of peptidoglycan, the major structural component of the bacterial cell wall. Moreover, these compounds are capable of inhibiting cell wall biosynthesis even in those cases where peptidoglycan is altered, thereby making them useful for treating Vancomycin Resistant Staphylococcus aureus (VRSA) strains. These compounds have shown efficacy against various Methicillin Resistant Staphylococcus (MRS) strains. However, as discussed earlier, these antibiotics are currently approved as parenteral compositions for skin related conditions.

Emergence of infections resistant to conventional ophthalmic and otic antibiotics is a serious issue. Therefore, there exists a need for safe and effective compositions for management of ophthalmic and otic infections which address the issue of antibiotic resistance. Although, various other antibiotics are well known for treating ophthalmic and otic infections, there is a need in the art for ophthalmic and otic compositions of lipoglycopeptides like Telavancin, Dalbavancin and Oritavancin which are active against the strains resistant to currently used antibiotics. Moreover, there is a need for safe and efficacious alternatives to glucocorticoids in treatment of inflammation associated with such infections. Therefore, it will be desirable to provide topical compositions of lipoglycopeptides like Telavancin, Dalbavancin and Oritavancin for ophthalmic and otic therapy. Further, it will be more desirable to provide topical compositions of these antibiotics which will also alleviate the inflammatory manifestations of the infection and still be devoid of si de-effects of glucocorticoids.

### SUMMARY OF THE INVENTION

Accordingly, the invention is based on topical compositions of lipoglycopeptide. The compositions of the present invention can be used to treat ophthalmic and otic infections and can be used prophylactically in post-operative management or in cases of ophthalmologic or otic trauma.

The invention relates to topical compositions of lipoglycopeptides selected from the group consisting of Telavancin, Dalbavancin and Oritavancin for treating the infections of eye and ear.

The compositions of the present invention deal with inflammation associated with eye and ear infections by additionally providing NSAID agents in combination with lipoglycopeptide antibiotics, thereby preventing the harmful side-effects of glucocorticoids.

The topical compositions of the invention comprise the lipoglycopeptides in an amount of about 0.001-5% w/v.

The topical compositions of the invention comprise the lipoglycopeptides in an amount of about 0.01-50 mg.

The compositions of the invention can be used to treat a variety of ophthalmic and otic infections, including those occurring due to bacterial strains resistant to conventionally used antibiotics.

### OBJECTIVE OF THE INVENTION

The main objective of the invention is to provide topical compositions comprising lipoglycopeptides and one or more pharmaceutically acceptable excipients.

Another objective of the invention is to provide topical compositions comprising lipoglycopeptides, one or more non-steroidal anti-inflammatory agent (NSAID) and one or more pharmaceutically acceptable excipients.

It is an objective of the invention to provide topical compositions comprising lipoglycopeptide for treating ophthalmic and otic infections.

It is an objective of the invention to provide topical compositions comprising lipoglycopeptide and one or more NSAID agent(s) for treating ophthalmic and otic infections.

It is an objective of the invention is to provide topical compositions of Telavancin and one or more pharmaceutically acceptable excipients.

It is an objective of the invention is to provide topical compositions of Dalbavancin and one or more pharmaceutically acceptable excipients.

It is an objective of the invention is to provide topical compositions of Oritavancin and one or more pharmaceutically acceptable excipients.

Still another objective of the present invention is to provide topical compositions comprising lipoglycopeptide selected from the group consisting of Telavancin, Dalbavancin and Oritavancin, one or more NSAID agent and one or more pharmaceutically acceptable excipients.

In yet another objective the invention provides topical compositions of lipoglycopeptides in an amount of about 0.001-5% w/v.

In yet another objective the invention provides topical compositions of lipoglycopeptides in an amount of about 0.01 to 50 mg.

### DETAILED DESCRIPTION OF THE INVENTION

As used herein, the term ‘lipoglycopeptide_ encompass Telavancin, Dalbavancin or Oritavancin.

As used herein, ‘Telavancin_, ‘Dalbavancin_ or ‘Oritavancin_ encompass free base, pharmaceutically acceptable salts, pharmacologically active metabolites thereof and their pharmaceutically acceptable salts, hydrates, its enantiomers or its racemates, unless otherwise noted.

The compositions of the invention may contain anti-inflammatory agents. The anti-inflammatory agents preferred in the present invention are non-steroidal anti-inflammatory agents (NSAIDs). NSAIDs which can be employed include without limitation Ketorolac, Bromfenac, Diclofenac, Flurbiprofen, Nepafenac free base, pharmaceutically acceptable salts, pharmacologically active metabolites hydrates, its enantiomers or its racemates unless otherwise noted. The concentrations of NSAIDs will depend on the actual agent used in the composition. The concentrations that can be employed are sufficient to reduce inflammation after topical application. The NSAIDs can be used in the concentration range of 0.001% to 5% w/w.

The invention provides topical compositions containing lipoglycopeptide antibiotics. Lipoglycopeptide antibiotics are selected from the group consisting of Telavancin, Dalbavancin and Oritavancin, which may optionally contain NSAIDs. Accordingly, the invention provides solutions, in-situ gels, lyophilized powders, liposomal compositions, emulsions, micro-emulsions dispersion, suspensions and nano-suspensions. The compositions of the invention can be used in treatment of eye or ear infections.

As an embodiment of the present invention, the compositions comprising lipoglycopeptide, optionally containing an NSAID are solutions for instilling in the eye or ear. Solutions further include excipients like buffering agents, chelating agents, preservatives, tonicity agents, solvents and combinations thereof.

Another embodiment of the present invention includes lyophilized powders of lipoglycopeptides, optionally comprises an NSAID. These powders can be reconstituted with suitable solvent, preferably water for injection before administration. The powders of the invention are prepared by lyophilization processes well-known in the art. As a further embodiment the powders after reconstitution may form a solution, dispersion, nano-suspension, micro-emulsion or liposomes for administration in eye or ear.

The compositions prepared by reconstituting powders may form in-situ gel after administration. As an embodiment the ocular residence time of the lipoglycopeptide compositions of the invention is improved by in-situ gelling systems. It is an embodiment of the present invention to provide in-situ gels or phase transition systems which are in the liquid form during instillation but, gel or solidify in the cul-de-sac subsequent to instillation. This phase transition from liquid to solid or semi-solid form is triggered by variety of factors which include pH of the tears, electrolytes present in the tears or surface temperature of the eye. Accordingly, it is embodiment of the present invention to include excipients which are responsible for in-situ gelling of compositions of lipoglycopeptides selected form the group consisting of Telavancin, Oritavancin and Dalbavancin, which may optionally include NSAID(s).

Sol-gel transition is triggered by change in surrounding temperature in thermo-reversible gels. The ideal critical temperature range for such systems is ambient and physiological temperature. The hydrogel gels are classified into three types based on the critical temperature as negative temperature sensitive, positive temperature sensitive and thermally reversible hydrogels. Negative temperature sensitive hydrogels have a lower critical solution temperature (LCST) whereas positive temperature sensitive gels have upper critical solution temperature (UCST). LCST polymers contract on heating above critical temperature and UCST polymers contract upon cooling below critical temperature. LCST polymers having critical temperature between ambient and physiologic temperature are preferred. Thermoreversible gels at elevated temperatures form random coils and upon cooling form a partial helix which is responsible for continuous network formation. In an embodiment of the present invention, temperature sensitive gelling polymers are incorporated in the compositions of lipoglycopeptides selected form the group consisting of Telavancin, Oritavancin and Dalbavancin which may optionally include NSAIDs. Such polymers without limitation include Poloxamer, cellulose derivative: methyl cellulose (MC), hydroxy propyl methyl cellulose (HPMC), ethyl (hydroxyethyl) cellulose, xyloglucan and others.

Ionic strength is other important factor in gelling of polymers. A change in ionic strength acts as a trigger for gelation. The rate of gelation is influenced by osmotic gradient across the gel surface. The osmolality of the solution, therefore, affects the rate of sol-gel transition, typically in eye. The aqueous polymer solutions form a gel in presence of mono or divalent cations present in tear fluids. The presence of Na, Mg and Ca cations and other electrolytes are responsible for triggering gelation in conjunctival cul-de-sac. It is an embodiment of the present invention to provide pharmaceutical compositions of lipoglycopeptides selected form the group consisting of Telavancin, Oritavancin and Dalbavancin which optionally include NSAIDs and polymers which gel in response to changes in ionic strength. Examples of such polymers without being bound to any limitations as such include Gellan gum, alginates, chitosan, fibrin, collagen, gelatin, hyaluronic acid, dextran, hydroxyethyl methacryate (HEMA), N-(2-hydroxy propyl)methacrylate (HPMA), N-vinyl-2-pyrrolidone (NVP), N-isopropylacrylamide (NIPAMM), vinyl acetate (VA), acryolic acid (AA), methacrylic acid (MAA), polyethylene glycol acrylate/methacrylate (PEGA/PEGMA), polyethylene glycol diacrylate/dimethacrylate (PEGDA/PEGDMA) and others.

Another mechanism to induce sol-gel transition is a change in pH. All the pH-sensitive polymers contain pendant acidic or basic groups that either accept or release protons in response to changes in environmental pH. Such polymers have a large number of ionizable groups. A hydrogel swells if the external pH increases in case of polymers having anionic groups while the converse is true in case of polymers having cationic groups. An embodiment of the present invention includes compositions of lipoglycopeptides selected form the group consisting of Telavancin, Oritavancin and Dalbavancin which optionally include NSAIDs and polymers which gel in response to change in pH. Polymers which are pH sensitive and can be a part of the present invention include without any limitations polyacrylic acid (carbopol or carbomer), pseudo latex e.g. (CAP Latex) cellulose acetate phthalate latex and others. A further embodiment includes a composition of Dalbavancin or Oritavancin which optionally include NSAIDs, in the liquid form having initial pH of 4.4 and undergoing coagulation with the rise in pH (7.7) due to contact with tear fluid. A change of 2.28 pH units leads to instantaneous gel-sol transition.

A further embodiment includes an oil-in-water emulsion ophthalmic or otic liquid composition of lipoglycopeptides selected from the group consisting of Telavancin Dalbavancin and Oritavancin. The emulsifying agents which can be used in the embodiment of the present invention include without limitation medium chain triglycerides, Pemulen. The oleaginous vehicle can includes without limitation castor oil, sesame oil, flaxseed oil, soybean oil.

Another embodiment of the present invention includes liposomal ophthalmic formulations of lipoglycopeptides selected from the group consisting of Telavancin, Dalbavancin and Oritavancin. The liposomal agents used can be selected from various ophthlamologically suitable lipids which include phosphatidyl choline, cholesterol, polyethylene glycol-12-glycerol dimethyacrylate and others.

As an embodiment of the present invention are lyophilized powders which form nano-suspension on reconstitution.

Successful ocular drug delivery is a major challenge due to the unique anatomy and physiology of eye which contains various types of barriers such as different layers of cornea, sclera and retina including blood aqueous and blood⁻retinal barriers, choroidal and conjunctival blood flow etc. These barriers cause a significant challenge for delivery of a drug alone or in a dosage form. Therefore, selection of appropriate excipients which are to be administered along with the active is an important concern. The selection of particular excipients and their concentrations is based not only on physical or chemical compatibility but also on biocompatibility with the sensitive ocular tissue.

The ophthalmic and otic compositions of the present invention can contain various pharmaceutically acceptable excipients. These include without limitation buffering agents, chelating agents, tonicity modifiers, viscosity enhancers, emulsifiers and surfactants, humectants and preservatives.

### i) Buffering Agents

Buffering agents play a vital role in maintaining pH of the compositions. Maintenance of adequate pH is necessary for stability and proper preservation of the compositions. Optimum pH of the ophthalmic solutions is also required for patient compliance and safety, particularly in case of ophthalmic products. The normal physiological pH of the tear fluid is 7.4. The pH of the compositions desired to be instilled in the eye. The compositions of the present invention include without limitation buffers such as phosphate buffers (potassium phosphate, di potassium hydrogen phosphate, potassium di hydrogen phosphate, disodium hydrogen phosphate, sodium dihydrogen phosphate), borate buffers (potassium borate, sodium borate), citrate buffers, tartarate buffers, acetate buffers, amino acids. The pH of the ophthalmic preparations of the current invention may also be modified by using a pH modifier. pH modifiers used in the present invention include hydrochloric acid and sodium hydroxide among others.

### ii) Chelating Agents

Chelating agents are used in ophthalmic compositions to enhance preservative effectiveness. While not intending to be limiting, some useful chelating agents for the purposes of this invention are edetate salts like edetate disodium edetate calcium disodium, edetate sodium, edetate trisodium, and edetate dipotassium. In preferred embodiment of this invention chelating agents like EDTA are used in ophthalmic preparations when the preservative used is Benzalkonium chloride.

iii) Tonicity Modifiers

The osmolarity of the lacrimal fluid is between 280 and 320 mOsm/kg which is dependent on the number of ions dissolved in the aqueous layer of the tear film. If ophthalmic preparation is hypotonic with respect to the ophthalmic environment, it will result into excessive tear secretion and irritation to the corneal epithelium. The osmotic pressure of the preparation must be same as that of normal saline solution. Therefore, tonicity adjusting agents employed in the compositions of the present invention without an intention of any limitation include sodium chloride, potassium chloride, buffer salts, dextrose, glycerine, propylene glycol, and mannitol.

### iv) Preservatives

Preservatives are useful in avoiding microbial contamination of ophthalmic or otic dosage forms. Examples of suitable preservatives employed in the present invention include without limitation, polyquatemium-1, benzalkonium chloride, sodium perborate, thimerosal, chlorobutanol, methyl paraben, propyl paraben, phenylethyl alcohol, edetate disodium, sorbic acid, or other agents known to those skilled in the art.

### v) Viscosity Modifiers

When administered as a drop, the ophthalmic preparations are washed away by tears due to inherent reflex mechanism, thereby diluting the concentration of the drug. The lacrimal drainage system responsible for this, leads the drug to nose and throat where it is absorbed into the general circulation, sometimes leading to serious systemic side effects. The otic compositions may drain to nasopharynx and upper throat through Eustachian tube. Therefore, it is an embodiment of the present invention to include viscosity imparting agent in the formulation to prolong the retention time of the drug solution at the target site. Examples without limitation include methyl cellulose, hydroxyl propyl cellulose, hydroxyl propyl methyl cellulose, polyvinyl alcohol and carbomers.

### vi) Emulsifiers and Surfactants

Surfactants are used in ophthalmic preparations to solubilize or disperse drugs in solution or dispersion. Surfactants also prevent the loss of drug to adsorption on the container walls. The compositions of the present invention without limitation may include sodium lauryl sulfate, polysorbates (tweens), tyloxapol. The compositions of the present invention may include emulsifying agents. Examples of such agents without limitation include Pemulenù, medium chain triglycerides.

vii) Humectants

Humectants are hygroscopic substances that keep the compositions moist. Humectants attract and retain the moisture in the air nearby via absorption, drawing the water vapor into the preparation. Without intending any limitation, humectants employed in the compositions in the compositions of the present invention include propylene glycols, glycerol, sorbitol, maltitol, xylitol.

The dosage forms of the present invention comprise of liquids, semi-solid and powders. The dosage forms of the present invention can be prepared by mixing the lipoglycopeptide antibiotics, NSAIDs (if present) and the pharmaceutically acceptable excipients in suitable solvents which without any limitation can comprise water, water for injection (WFI), sugar solution etc. The pH is adjusted, if required. The resultant compositions are then sterilized using suitable techniques. The compositions can be freeze dried to obtain lyophilized powders.

The present invention is further described by way of the following non-limiting examples.

TABLE 1
Lipoglycopeptide compositions for ophthalmic or otic use

| Ingredient | E1* | E2* | E3* | E4* | E5* | E6* | E7* | E8* | E9* |
|---|---|---|---|---|---|---|---|---|---|
| Active** | 0.5 | 0.5 | 0.5 | 0.5 | 0.5 | 0.5 | 0.5 | 0.5 | 0.5 |
| Edetate | 0.015 | — | — | 0.06 | 0.06 | — | — | — | — |
| diSodium | | | | | | | | | |
| Disodium | 0.01-5 | 0.01-5 | — | — | — | — | — | — | 0.01-5 |
| hydrogen | | | | | | | | | |
| phosphate | | | | | | | | | |
| Sodium | — | — | — | — | — | 0.01-2 | — | 0.01-2 | — |
| citrate | | | | | | | | | |
| dihydrate | | | | | | | | | |
| Citric acid | — | — | — | — | — | — | — | — | 0.001-1 |
| Sodium | 0.01-2 | 0.01-2 | 0.01-2 | 0.01-2 | 0.01-2 | 0.01-2 | 0.01-2 | — | — |
| chloride | | | | | | | | | |
| Mannitol | — | — | — | — | — | — | — | — | 0.4 |
| Dextrose | — | — | — | — | — | — | — | 0.16 | — |
| Carbomer | — | — | 0.01-5 | 0.01-5 | — | — | — | — | 0.01-5 |
| Poloxamer | — | — | — | — | — | 5 | — | — | — |
| Xanthan | — | — | — | — | — | — | 0.1 | — | — |
| gum | | | | | | | | | |
| HPMC | — | — | — | — | — | — | — | — | 0.05 |
| Methyl | — | — | — | — | — | — | 0.25 | — | — |
| Cellulose | | | | | | | | | |
| Gellan gum | — | — | — | — | — | — | — | 0.5 | — |
| Polysorbate | — | — | 0.3 | — | — | — | — | — | — |
| Tyloxapol | — | — | — | — | 0.05 | — | — | — | — |
| Benzalkonium | — | 0.006 | 0.006 | 0.008 | 0.005 | — | — | 0.006 | — |
| chloride | | | | | | | | | |
| Sodium | — | — | — | — | — | 0.008 | 0.008 | — | — |
| perborate | | | | | | | | | |
| Boric acid | — | — | — | — | — | 0.2 | — | — | — |
| Hydrochloric | Add^{#} | Add^{#} | Add^{#} | Add^{#} | Add^{#} | — | — | — | — |
| acid/Sodium | | | | | | | | | |
| Hydroxide | | | | | | | | | |
| Water for | q.s. | q.s. | q.s. | q.s. | q.s. | q.s. | q.s. | q.s. | q.s. |
| Injection | 100 | 100 | 100 | 100 | 100 | 100 | 100 | 100 | 100 |
| (WFI) | | | | | | | | | |

*Concentration in % w/v
**Active is selected from a group consisting of Telavancin, Oritavancin and Dalbavancin
^{#}Add to adjust the pH to 7.4

The general manufacturing procedure steps for the compositions from Table 1 is as below—
i. Mix buffers (Disodium hydrogen phosphate, Sodium citrate dihydrate); buffering agents (citric acid, boric acid); tonicity modifiers (sodium chloride, mannitol, dextrose); gellants (Carbomer, xanthan gum, Hydroxypropylmethylcellulose, poloxamer, gellan gum, methylcellulose); surfactants (polysorbate, tyloxapol) with 50% batch quantity of WFI.
ii. Stir the mixture of step (i) to obtain a clear solution.
iii. Add the Active to the solution of step (ii) under continuous stirring to a obtain a clear solution
iv. Preservatives (benzalkonium chloride and sodium perborate) if present, are added to the solution of step (iii).
v. The pH of the solution of step (iv) is adjusted to 7.4 using hydrochloric acid or sodium hydroxide, if required.
vi. Filter the solution of step (v) thorough 0.21 i m filter.
vii. The compositions of step (vi), may optionally be freeze dried to obtain lyophilized powders.

TABLE 2
Lipoglycopeptide compositions with NSAID for ophthalmic or otic use

| Ingredient | E1* | E2* | E3* | E4* | E5* | E6* | E7* | E8* | E9* |
|---|---|---|---|---|---|---|---|---|---|
| Active** | 0.5 | 0.5 | 0.5 | 0.5 | 0.5 | 0.5 | 0.5 | 0.5 | 0.5 |
| NSAID*** | 0.001-5 | 0.001-5 | 0.001-5 | 0.001-5 | 0.001-5 | 0.001-5 | 0.001-5 | 0.001-5 | 0.001-5 |
| Edetate | 0.015 | — | — | 0.06 | 0.06 | — | — | — | — |
| diSodium | | | | | | | | | |
| Disodium | 0.01-5 | 0.01-5 | — | — | — | — | — | — | 0.01-5 |
| hydrogen | | | | | | | | | |
| phosphate | | | | | | | | | |
| Sodium | — | — | — | — | — | 0.01-2 | — | 0.01-2 | — |
| citrate | | | | | | | | | |
| dihydrate | | | | | | | | | |
| Citric acid | — | — | — | — | — | — | — | — | 0.001-1 |
| Sodium | 0.01-2 | 0.01-2 | 0.01-2 | 0.01-2 | 0.01-2 | 0.01-2 | 0.01-2 | — | — |
| chloride | | | | | | | | | |
| Mannitol | — | — | — | — | — | — | — | — | 0.4 |
| Dextrose | — | — | — | — | — | — | — | 0.16 | — |
| Carbomer | — | — | 0.01-5 | 0.01-5 | — | — | — | — | 0.01-5 |
| Poloxamer | — | — | — | — | — | 5 | — | — | — |
| Xanthan | — | — | — | — | — | — | 0.1 | — | — |
| gum | | | | | | | | | |
| HPMC | — | — | — | — | — | — | — | — | 0.05 |
| Methyl | — | — | — | — | — | — | 0.25 | — | — |
| Cellulose | | | | | | | | | |
| Gellan gum | — | — | — | — | — | — | — | 0.5 | — |
| Polysorbate | — | — | 0.3 | — | — | — | — | — | — |
| Tyloxapol | — | — | — | — | 0.05 | — | — | — | — |
| Benzalkonium | — | 0.006 | 0.006 | 0.008 | 0.005 | — | — | 0.006 | — |
| chloride | | | | | | | | | |
| Sodium | — | — | — | — | — | 0.008 | 0.008 | — | — |
| perborate | | | | | | | | | |
| Boric acid | — | — | — | — | — | — | 0.2 | — | — |
| Hydrochloric | Add^{#} | Add^{#} | Add^{#} | Add^{#} | Add^{#} | — | — | — | — |
| acid/Sodium | | | | | | | | | |
| Hydroxide | | | | | | | | | |
| Water for | q.s. | q.s. | q.s. | q.s. | q.s. | q.s. | q.s. | q.s. | q.s. |
| Injection | 100 | 100 | 100 | 100 | 100 | 100 | 100 | 100 | 100 |
| (WFI) | | | | | | | | | |

*Concentration in % w/v
**Active is selected from a group consisting of Telavancin, Oritavancin and Dalbavancin
***NSAID is selected from Nepafenac, Ketorolac tromethamine, Bromfenac, Diclofenac sodium, Flurbiprofen
^{#}Add to adjust the pH to 7.4

The general manufacturing procedure steps for the compositions from Table 2 is as below—
i) Mix buffers (Disodium hydrogen phosphate, Sodium citrate dihydrate); buffering agents (citric acid, boric acid); tonicity modifiers (sodium chloride, mannitol, dextrose); gellants (Carbomer, xanthan gum, Hydroxypropylmethylcellulose, poloxamer, gellan gum, methyl cellulose); surfactants (polysorbate, tyloxapol) with 50% batch quantity of WFI.
ii) Stir the mixture of step (i) to obtain a clear solution.
iii) Add the Active to the solution of step (ii) under continuous stirring to a obtain a clear solution
iv) Add the NSAID under continuous stirring to the solution of step (iii).
v) Preservatives (benzalkonium chloride and sodium perborate) if present, are added to the solution of step (iv).
vi) The pH of the solution of step (iv) is adjusted to 7.4 using hydrochloric acid or sodium hydroxide, if required.
vii) Filter the solution of step (vi) thorough 0.2 i m filter, if it ̆s a dispersion (in case of Nepafenac) sterilization is done by radiation.
viii) The compositions of step (vii), may optionally be freeze dried to obtain lyophilized powders.

TABLE 3
Nano-suspension of Lipoglycopeptide

| Ingredient | E1* | E2* | E3* |
|---|---|---|---|
| Active** | 0.5 | 0.5 | 0.5 |
| Sodium lauryl | — | 0.1 | — |
| sulfate | | | |
| Polysorbate | 0.1 | — | 0.3 |
| Mannitol | — | 16.7 | — |
| Glycerol 85% | — | — | 16.7 |
| Water for | q.s. 100 | q.s. 100 | q.s. 100 |
| Injection (WFI) | | | |

*Concentration in % w/v
**Active is selected from a group consisting of Telavancin, Oritavancin and Dalbavancin.

The manufacturing procedure for powder for nano-suspension is described below—
i) Prepare a concentrated solution of surfactant (Sodium Lauryl Sulfate, polysorbate) and tonicity modifying agent; if present (mannitol, glycerol 80%) for wetting.
ii) Add additional quantity of Water for Injection to the solution of step (i).
iii) Add the active into the solution of step (ii) by stirring.
iv) Pass the coarsely dispersed suspension through a continuously operating homogenizer at room temperature.
v) Makeup the volume of the suspension of step (iv) with WFI.
vi) Sterilize the suspension of step (v) by radiation sterilization.
vii) The composition of the step (vi) is then freeze dried to get lyophilized powder for reconstitution.

TABLE 4
Micro-emulsion of Lipoglycopeptides

| Ingredient | E1* | E2* |
|---|---|---|
| Active** | 0.5 | 0.5 |
| Medium chain Triglyceride | 0.7 | — |
| Pemulen | — | 0.05 |
| Vitamin E TPGS | 2 | — |
| Polysorbate | 1 | — |
| Glycerin | 2 | 2.2 |
| Castor oil | — | 1.25 |
| Hydrochloric acid/Sodium | Added | Added |
| Hydroxide | | |
| Water for Injection | q.s. 100 | q.s. 100 |

*Concentration in % w/v
**Active is selected from a group consisting of Telavancin, Oritavancin and Dalbavancin.

The procedure for preparation of micro-emulsion is—
i) Weigh and heat the components of oily phase (medium chain triglyceride, Vitamin E TPGS, Pemulen, Polysorbate) with the active in a water bath.
ii) Stir the mixture of step (i) until a clear, slightly viscous solution is obtained.
iii) Weigh the components of aqueous phase (glycerin) and dissolve in water until a clear solution is obtained.
iv) Two phases of step (ii) and (iii) are mixed and gently stirred with a magnet
v) The pH of the solution of step (iv) is adjusted between 5.5-7.2 using dilute solution of hydrochloric acid or sodium hydroxide.
vi) Sterilize the composition by radiation sterilization.

TABLE 5
Powder for Liposomal composition of Lipoglycopeptides
Example 5a:

| Ingredient | % w/w |
|---|---|
| Active* | 0.5 |
| Hypromellose (HPMC) | 3 |
| Monobasic sodium phosphate | 10 |
| Dibasic sodium phosphate | 3 |
| Polysorbate 80 | 0.5 |
| Sodium chloride | 2.5 |
| Benzalkonium chloride | 0.2 |
| Polyethylene glycol-12-glycerol | 10 |
| dimethacrylate (PEG-12-GDM) | |
| NaOH or HCl to adjust | pH 5.0 to 7.5 |
| Water for Injection (WFI) | q.s. up to 100 |

*Active is selected from a group consisting of Telavancin, Oritavancin and Dalbavancin.

Following is the process for making powder for liposomal formulation—
i) Place 40% of the final volume of distilled water in beaker and heat it to 70 éC.
ii) Add HPMC to the beaker in step (i) and stop mixing until reaching room temperature and it becomes a clear and homogeneous mixture.
iii) Autoclave the solution of step (ii) and once sterile allows it to reach room temperature while stirring.
iv) Place in another beaker 40% of the final volume of distilled water. Add and mix until completely dissolved one by one the following reagents: a) Sodium phosphate monobasic b) Sodium phosphate dibasic c) Sodium chloride d) Polysorbate 80
v) In 10% of the remaining volume of water, add the benzalkonium chloride at 50% and mix until completely incorporated. Once dissolved, add this new solution to the above solution of step (iv)
vi) Mix the sterile solution of HPMC with the other sterile solution containing the salts and the preservative benzalkonium chloride and mix until getting a clear homogeneous mixture.
vii) Dissolve the active in oily phase (PEG-12-GDM).
viii) Liposome preparation entails merely mixing the lipid of step (vii) with an aqueous solution of step (i-iii)
ix) Add the Liposomes to this mixture and stir during 15 minutes with a magnetic stirrer to obtain a final suspension.
x) Sterilize the dispersion of step (ix) by radiation sterilization.
xi) Freeze dry the dispersion of step (x) to get the lyophilized powder for reconstitution.

### Example 5b

| Ingredient | % w/w |
|---|---|
| Active* | 0.5 |
| Phosphatidylcholine | 12 |
| Cholesterol | 1.7 |
| Sodium deoxycholate | 1 |
| Chloroform/diethyl | 30 |
| ether (1:3) | |
| Water for Injection | 10 |
| (WFI) | |
| Saccharose solution | q.s. |
| (5% w/v) | |

*Active is selected from a group consisting of Telavancin, Oritavancin and Dalbavancin.

Following is the process for making powder for liposomal formulation—
i) Prepare a mixture of phosphatidylcholine, cholesterol, sodium deoxycholate (12:1.7:1, w/w) and dissolve it in chloroform/diethyl ether solution.
ii) Add aqueous solution of active to step (i) and sonicate for 10 minutes to form an emulsion (w/o).
iii) Evaporate the organic solvents form emulsion of step (ii) using a rotary evaporator under vacuum at 37 éC and add saccharose solution (5% w/v).
iv) Sterilize the dispersion of step (iii) by radiation sterilization.
v) Freeze dry the filltrate of step (iv) to get the lyophilized powder for reconstitution.

TABLE 6
Powder for Gel based composition of Lipoglycopeptides

| Ingredient | E1* | E2* |
|---|---|---|
| Active** | 0.5 | 0.5 |
| Xanthan gum | 0.5 | 0.8 |
| Sorbitol | 25 | 4 |
| Sodium edetate | 0.1 | 0.05 |
| Benzalkonium chloride | 0.008 | — |
| Hydrochloric acid/Sodium Hydroxide | — | Added |
| Water for Injection (WFI) | q.s. 100 | q.s. 100 |

*Concentration in % w/v
**Active is selected from a group consisting of Telavancin, Oritavancin and Dalbavancin.

The process for preparation of the gel based formulations is described below—
i) Dissolve Xanthan gum in a portion of WFI.
ii) Add active, sorbitol and Sodium Edetate to the remaining amount WFI,
iii) Then, combine the solution of step (ii) and step (i) and homogenize it.
iv) Optionally, add benzalkonium chloride, to the preparation of step (iii) and mix properly.
v) Adjust the pH value to 7.4 with 1N HCl or NaOH.
vi) Sterilize the dispersion of step (v) by radiation sterilization.
vii) Freeze dry the gel to get the lyophilized powder for reconstitution.

The invention has been described herein by reference to certain preferred embodiments. However, as obvious variations thereon will become apparent to those skilled in the art, the invention must not be as limited thereto.

1. A topical pharmaceutical composition comprising a lipoglycopeptide component selected from the group consisting of Telavancin, Oritavancin and Dalbavancin; optionally a non-steroidal anti-inflammatory drug; and one or more pharmaceutically acceptable excipients.

2. The topical composition of claim 1 wherein the non-steroidal anti-inflammatory drug is selected from the group consisting of Ketorolac, Bromfenac, Diclofenac, Flurbiprofen and Nepafenac or pharmaceutically acceptable salts thereof.

3. The topical composition of claim 1 wherein the composition is administered to eye or ear of a patient to treat infection.

4. The topical composition of claim 1 wherein, the composition is solution, dispersions, suspension, nano-suspensions, micro-emulsion, in-situ gel or liposomal formulation.

5. The topical composition of claim 1, wherein the pharmaceutically acceptable excipients are buffering agents, acidifying agents, chelating agents, tonicity modifiers, preservatives, suspending agents, emulsifying agents, surfactants, ionic detergents, gelling agents, lipids, oils and humectants, or combinations thereof.

6. The topical composition of claim 1 wherein, the composition is lyophilized powder suitable for reconstitution.

7. The powder of claim 6 wherein the powder comprises from about 0.01 mg to about 50 mg of lipoglycopeptides.

8. The topical composition of claim 6 wherein the lyophilized powder on reconstitution forms solution, dispersion, suspension, nano-suspension, in-situ gel, or liposomal formulation.

9. The topical composition of claim 6, wherein, the composition is prepared by a process comprising a) adding pharmaceutically acceptable excipients and lipoglycopeptides to water for injection; b) sterilizing the mixture of step (a); and c) freeze drying the mixture of step (b) to obtain lyophilized powder.
